(19) United States
(12) Patent Application Publication
(10) Pub. No.: US 2021/0401766 A1
(43) Pub. Date: Dec. 30, 2021

(54) Method of Using Cannabinoids Encapsulated in Phospholipid Carriers for Transmucosal and Transdermal Administration

(71) Applicant: NuVessl, Inc., Calgary (CA)

(72) Inventors: Tanya Rhodes, Largo, FL (US); Deborah Duffey, Safety Harbor, FL (US)

(73) Assignee: NuVessl, Inc., Calgary (CA)

(21) Appl. No.: 17/466,455

(22) Filed: Sep. 21, 2021

Related U.S. Application Data
(63) Continuation of application No. 16/560,491, filed on Sep. 4, 2019.
(60) Provisional application No. 62/727,996, filed on Sep. 6, 2018.

(51) Int. Cl. A61K 9/70; A61K 9/00; A61K 9/06; A61K 9/127; A61K 31/05; A61K 36/185; A61K 47/10; A61K 9/51; A61K 31/352; A61K 9/107; A61K 31/353; A61K 47/24; A61K 47/44

(52) U.S. Cl. CPC A61K 9/7023; A61K 9/006; A61K 9/0014; A61K 9/06; A61K 9/127; A61K 31/05; A61K 47/44; A61K 47/10; A61K 9/51; A61K 31/352; A61K 9/1075; A61K 31/353; A61K 47/24; A61K 36/185

(57) ABSTRACT

A method of administering a composition having Cannabis sativa-derived substances nano-encapsulated in phospholipid vesicles for transmucosal and transdermal delivery is disclosed. A Cannabis sativa-derived formulation for transmucosal and transdermal delivery comprising Cannabis sativa-derived compounds nanoencapsulated in phospholipid-based vesicles is also disclosed. Cannabis sativa-derived extracts have enhanced bioavailability when encapsulated in nanosized phospholipid vesicles prior to administration to a subject as compared to non-encapsulated cannabinoids. A method of encapsulating cannabis-derived compounds in nanosized phospholipid vesicles is also disclosed

### CROSS REFERENCE TO RELATED APPLICATIONS

This application is a continuation of U.S. application Ser. No. 16/560,491, filed Sep. 4, 2019, which claims the benefit of U.S. Provisional Application No. 62/727,996 filed Sep. 6, 2018, which is incorporated by reference into this utility patent application.

### STATEMENT REGARDING FEDERALLY SPONSORED RESEARCH OR DEVELOPMENT

Not applicable.

### TECHNICAL FIELD OF INVENTION

The present invention relates to encapsulation of cannabinoid compounds, compositions and uses thereof.

### BACKGROUND OF THE INVENTION

During most of the 20th century, the United States Food and Drug Administration (“FDA”) questioned the safety of cannabis due to its potential for abuse, as well as the adverse cardiovascular, reproductive and pulmonary effects associated with inhalation of any type of smoke. In 1999, the Office of National Drug Control funded a study by the Institute of Medicine to evaluate medicinal uses of cannabis. The outcome of this 1999 study was a recommendation to test alternative cannabinoid delivery systems for medicinal use (other than inhalation of cannabis smoke).

Since then, a number of pharmaceutical companies have analyzed the whole plant extract (and cannabidiol (CBD) and tetrahydrocannabinol (THC)) for possible medicinal uses. Some of these extracts have been approved in Canada for neuropathic pain in multiple sclerosis and as an analgesic for cancer pain. It is currently in phase III trials in the US for a cancer pain indication.

Many studies are underway looking at the individual components of CBD and THC as well as the many specific isolates, to better define the mechanisms of action with respect to the different receptors in the body. According to the United States National Institute of Health (“NIH”) there are more than 60 systematic reviews discussing the safety, toxicology, potency, and therapeutic potential of cannabinoids. Preclinical trials over the past four decades have found that the cannabinoids show promise as an anti-inflammatory, neuroprotectant, analgesic, anti-tumoral agent and anti-psychotic. Cannabidiol has also been found to be effective for control of seizures associated with certain types of epilepsy. Certain cannabinoids, like CBD, have been well-studied and are well tolerated and safe in humans, even at high doses over a period of time.

Cannabis sativa has over 483 known compounds, over 60 of which are classified as cannabinoids, many of which have mental and physical effects, including tetrahydrocannabinol (THC) and cannabidiol (CBD). Cannabidiol (CBD) is one of the most prevalent chemical compounds in the cannabis plant. Among the other cannabis-derived compounds are a variety of terpenes. These terpenes are oils secreted from the plant which give the plant its characteristic odor. Terpenes produce physical effects similar to those seen with CBD.

Hemp is a variety of the Cannabis sativa plant species that is grown specifically for the industrial uses of its derived products. Although cannabis used as a drug and hemp for industrial use both derive from the species Cannabis sativa and contain tetrahydrocannabinol (THC), the strains are distinct and have unique phytochemical compositions and uses. Hemp has lower concentrations of THC and higher concentrations of cannabidiol (CBD), which decreases or eliminates its psychoactive effects. The higher concentrations of cannabidiol and lower concentrations of THC make hemp an ideal source for extraction of CBD.

THC is the primary compound associated with cannabis psychoactive effects, while CBD does not appear to have psychoactive effects. Unlike THC, CBD is non-psychoactive, but still has an effect on the body. The THC portion of the plant is the psychoactive ingredient and therefore progressing the use of medical CBD alone is viewed as viable.

When considering the medical use of a Cannabidiol (CBD) substance, the bioavailability of the CBD substance is the degree and rate at which the CBD is absorbed into the body or is made available at the site of physiological activity. This degree and rate of absorption is an essential determining factor as to the efficacy of the drug. There has been very little comparable testing done on the bioavailability of CBD through the different modes of administration in humans, and it is critical to gain an understanding of the pharmacokinetics of CBD to ensure that it is controlled and understood for each route of administration and any specific indications.

Even though oral administration of CBD has shown anecdotal positive effects, the bioavailability of oral CBD administrated substances appears to be very low (˜6%-15%) due to the “first pass” metabolism.” There is also been shown to be a great variation from individual to individual in the absorption rates for oral CBD administrated substances, with the effects generally lasting between 5-8 hours resulting in a lower re-medication rate. Sublingual (under the tongue) and buccal (cheek or gums) have shown similar bioavailability results to oral administration. While sublingual or buccal is not as fast acting as smoking/vaporizing, it is considered a much safer option than smoking.

Studies have shown oromucosal bioavailability to be approximately 35% bioavailability. With the exception of intravenous (IV) application, smoking and vaporizing shows the highest bioavailability in the shortest amount of time and as such becomes the target standard to achieve by a different route of administration (2%-56% with average around 40%); however, the length of time CBD remains in the body via this route is only around 3 hours. Smoking and vaporizing has historically been the route of administration for experienced users, but is not considered the most acceptable route of administration due to exposure to the adverse health consequences of inhaling smoke in the lungs. These adverse health impacts that arise with smoking and vaporizing make it an unhealthy choice for the administration of a CDB substance.

Recent studies have suggested a clear role for CBD as a medical therapy for many disorders; however, the route of administration severely impacts the bioavailability of the CBD, and in all acceptable cases the bioavailability remains low. A route of administration that is safe and controlled, that reaches the bloodstream as quickly as smoking, has the highest peak value, and remains in the bloodstream for the longest time is needed.

### SUMMARY OF THE INVENTION

Disclosed herein is a method for administration of a Cannabis sativa-derived substance in an intraoral transmucosal and/or transdermal delivery protocol having the steps of: providing one or more phospholipid vesicles in a first phospholipid solution having 75-89% ethanol (w/w), 10-20% phospholipids (w/w), and 1-5% water (w/w), and encapsulating one or more Cannabis sativa-derived substances in the one or more phospholipid vesicles in the first phospholipid solution; the encapsulated substances in the phospholipid vesicles being an initial cannabis preparation. The initial cannabis preparation is combined with a predetermined amount of ethanol to produce an ethanol-diluted cannabis preparation having phospholipid vesicles being predominately sized less than 10 nm, the size of vesicles in the ethanol-diluted cannabis preparation being controlled predominately by the amount of ethanol added to the initial cannabis preparation.

As disclosed herein, the ethanol-diluted cannabis preparation is combined with a predetermined quantity of water to produce increased-sized phospholipid vesicles in an encapsulated cannabis preparation. The increased-sized phospholipid vesicles in the encapsulated cannabis preparation are predominately sized between 25-200 nm having a concentration of 0.01% to 1.0% of the Cannabis sativa-derived substance encapsulated therein. The size of the increased-sized phospholipid vesicles in the encapsulated cannabis preparation is controlled predominately by the amount of water added to the ethanol-diluted cannabis preparation. The encapsulated cannabis preparation, in an unrefined form without vesicle size reduction by mechanical processing, is combined with one or more excipient ingredients suitable for intraoral transmucosal or transdermal application to yield a cannabis formulation for intraoral transmucosal or transdermal administration and administered as a quantity of the cannabis formulation having 1 mg to 100 mg of encapsulated Cannabis sativa-derived substances to a subject in need of treatment.

In the disclosed method, the Cannabis sativa-derived substance may be selected from cannabidiol (CBD), tetrahydrocannabinol (THC), Cannabis sativa-derived terpenes, Cannabis sativa-derived flavonoids, a whole plant extract of Cannabis sativa, and combinations thereof. Optionally, the Cannabis sativa-derived substance is cannabidiol (CBD), tetrahydrocannabinol (THC), or a combination of cannabidiol (CBD) and tetrahydrocannabinol (THC).

In the disclosed method, Cannabis sativa-derived substances are combined with the first phospholipid solution in a concentration range from 1 part Cannabis sativa-derived substance to 10 parts of the first phospholipid solution to 10 parts Cannabis sativa-derived substance to 1 part first phospholipid solution. The predetermined quantity of ethanol combined with the initial cannabis preparation has a ratio of 1:1 to 1:10 parts of initial cannabis preparation to ethanol. The predetermined quantity of water combined with the ethanol-diluted cannabis preparation has a ratio of 1:5 to 1:25 parts of ethanol-diluted cannabis preparation to water. phosphatidylcholine (PC) is at least 50% of the phospholipid total in the first phospholipid solution.

Also disclosed herein is method for intraoral transmucosal administration of a Cannabis sativa-derived substance having the steps of: providing one or more phospholipid vesicles in a first phospholipid solution having 75-89% ethanol (w/w), 10-20% phospholipids (w/w), and 1-5% water (w/w), and encapsulating one or more Cannabis sativa-derived substances in the one or more phospholipid vesicles in the first phospholipid solution, the encapsulated substances in the phospholipid vesicles being an initial cannabis preparation, wherein the Cannabis sativa-derived substances are combined with the first phospholipid solution in a concentration range from 1 part Cannabis sativa-derived substance to 10 parts of the first phospholipid solution to 10 parts Cannabis sativa-derived substance to 1 part first phospholipid solution. The initial cannabis preparation is combined with a predetermined amount of ethanol to produce an ethanol-diluted cannabis preparation having phospholipid vesicles being predominately sized less than 10 nm, the predetermined quantity of ethanol combined with the initial cannabis preparation having a ratio of 1:1 to 1:10 parts of initial cannabis preparation to ethanol, the size of vesicles in the ethanol-diluted cannabis preparation being controlled predominately by the amount of ethanol added to the initial cannabis preparation.

As disclosed herein, the ethanol-diluted cannabis preparation is combined with a predetermined quantity of water to produce increased-sized phospholipid vesicles in an encapsulated cannabis preparation, the size of the increased-sized phospholipid vesicles in the encapsulated cannabis preparation being controlled predominately by the amount of water added to the ethanol-diluted cannabis preparation. The encapsulated cannabis preparation in an unrefined form without vesicle size reduction by mechanical processing, is combined with one or more excipient ingredients suitable for intraoral transmucosal application to yield a cannabis formulation for intraoral transmucosal administration, and a quantity of the cannabis formulation having 1 mg to 100 mg of encapsulated Cannabis sativa-derived substances is administered to an intraoral mucosa of a subject in need of treatment.

In the disclosed method, the increased sized phospholipid vesicles in the encapsulated cannabis preparation are predominately sized between 25-200 nm. The Cannabis sativa-derived substance is selected from cannabidiol (CBD), tetrahydrocannabinol (THC), Cannabis sativa-derived terpenes, Cannabis sativa-derived flavonoids, a whole plant extract of Cannabis sativa, and combinations thereof. The predetermined quantity of water combined with the ethanol-diluted cannabis preparation has a ratio of 1:5 to 1:25 parts of ethanol-diluted cannabis preparation to water, and the phospholipid vesicles in the encapsulated cannabis preparation have a concentration of 0.01% to 1.0% of the Cannabis sativa-derived substance encapsulated therein. The cannabis formulation may be applied to the intraoral mucosa as a liquid drop, liquid spray, aerosol, liquid shot, gel, paste, lozenge, gum, gummy candy, hard candy, orally dissolving strip, tablet, or swish and swallow preparation.

Also disclosed herein is a method for transdermal administration of a Cannabis sativa-derived substance comprising the steps of: providing one or more phospholipid vesicles in a first phospholipid solution having 75-89% ethanol (w/w), 10-20% phospholipids (w/w) with phosphatidylcholine (PC) being at least 50% of the phospholipid total in the first phospholipid solution, and 1-5% water (w/w), and encapsulating one or more Cannabis sativa-derived substances selected from cannabidiol (CBD), tetrahydrocannabinol (THC), Cannabis sativa-derived terpenes, Cannabis sativa-derived flavonoids, a whole plant extract of Cannabis sativa, and combinations thereof, in one or more phospholipid vesicles in the first phospholipid solution, the encapsulated substances in the phospholipid vesicles being an initial cannabis preparation. The initial cannabis preparation is combined with a predetermined amount of ethanol to produce an ethanol-diluted cannabis preparation having phospholipid vesicles being predominately sized less than 10 nm, the size of vesicles in the ethanol-diluted cannabis preparation being controlled predominately by the amount of ethanol added to the initial cannabis preparation.

As disclosed herein, the ethanol-diluted cannabis preparation is combined with a predetermined quantity of water to produce increased-sized phospholipid vesicles in an encapsulated cannabis preparation, the increased sized phospholipid vesicles in the encapsulated cannabis preparation being predominately sized between 25-200 nm, and the size of the increased-sized phospholipid vesicles in the encapsulated cannabis preparation being controlled predominately by the amount of water added to the ethanol-diluted cannabis preparation. The encapsulated cannabis preparation in an unrefined form without vesicle size reduction by mechanical processing, is combined with one or more excipient ingredients suitable for transdermal application to yield a cannabis formulation for transdermal administration, and a quantity of the cannabis formulation having 1 mg to 100 mg of encapsulated Cannabis sativa-derived substances is administered to an epidermal area of a subject in need of treatment.

In the disclosed method, Cannabis sativa-derived substance is cannabidiol (CBD), tetrahydrocannabinol (THC), or a combination of cannabidiol (CBD) and tetrahydrocannabinol (THC). The Cannabis sativa-derived substance are combined with the first phospholipid solution in a concentration range from 1 part Cannabis sativa-derived substance to 10 parts of the first phospholipid solution to 10 parts Cannabis sativa-derived substance to 1 part first phospholipid solution, and the phospholipid vesicles in the encapsulated cannabis preparation have a concentration of 0.01% to 1.0% of the Cannabis sativa-derived substance encapsulated therein. The predetermined quantity of ethanol combined with the initial cannabis preparation has a ratio of 1:1 to 1:10 parts of initial cannabis preparation to ethanol. The predetermined quantity of water combined with the ethanol-diluted cannabis preparation has a ratio of 1:5 to 1:25 parts of ethanol-diluted cannabis preparation to water. The cannabis formulation may be applied to the epidermis in a cream, lotion, ointment, wax, topically applied spray, gel, balm or transdermal patch.

The present invention is a composition formulated from a Cannabis sativia-derived substance for transmucosal and transdermal delivery using encapsulation in phospholipid carriers. Cannabis sativa-derived substances for encapsulation are selected from cannabidiol (CBD), tetrahydrocannabinol (THC), Cannabis sativa-derived terpenes, Cannabis sativa-derived flavonoids, a whole plant extract of Cannabis sativa, and combinations thereof. The Cannabis sativa-derived substance may be cannabidiol (CBD). The Cannabis sativa-derived substance may be tetrahydrocannabinol (THC). The Cannabis sativa-derived substance may be a combination of cannabidiol (CBD) and tetrahydrocannabinol (THC). The Cannabis sativa-derived substance may be a whole plant extract of Cannabis sativa.

The composition formulated for transmucosal or transdermal delivery of Cannabis sativa derived substances in the present invention is formulated to be encapsulated in a phospholipid delivery system. For the present invention, one or more Cannabis sativa-derived substance is nanoencapsulated in phospholipid vesicles having phospholipids, ethanol and water. The phospholipid vesicles having the encapsulated Cannabis sativa-derived substance are sized from 25 nm to 200 nm. Phosphatidylcholine comprises at least 50% by weight of the phospholipids in the phospholipid vesicles. The concentration range of Cannabis sativa-derived substance encapsulated in the phospholipid vesicles is from 0.01% to 1.0% w/w.

For transmucosal administration, the phospholipid vesicles with the nanoencapsulated Cannabis sativa-derived substance are administered to the oral cavity of a subject for transmucosal uptake of the Cannabis sativa-derived substance. Transmucosal delivery can be via liquids such as drops, sprays, aerosols or shots, gels, pastes, lozenges, gums, gummy candies, hard candies, orally dissolving strips, tablets, swish and swallow preparations, or other form suitable for contact to oral mucosa. For transdermal administration, the phospholipid vesicles with the nanoencapsulated Cannabis sativa-derived substance are applied to the epidermis of a subject for transdermal uptake of the Cannabis sativa-derived substance. Transdermal delivery can be via a topically applied preparation, such as a cream, lotion, ointment, wax, topically applied spray, gel, balm, transdermal patch or other transdermal application means.

### BRIEF DESCRIPTION OF THE FIGURES

FIG. 1 is a graph showing a comparison of serum levels of encapsulated vs. non-encapsulated CBD against historical data.

### DETAILED DESCRIPTION

Disclosed herein is a composition comprising Cannabis sativa-derived substances encapsulated in a phospholipid delivery system. The Cannabis sativa-derived substances for encapsulation are selected from cannabidiol (CBD), tetrahydrocannabinol (THC), Cannabis sativa-derived terpenes, Cannabis sativa-derived flavonoids, a whole plant extract of Cannabis sativa, and combinations thereof.

Cannabis sativa-derived substances for encapsulation may be cannabidiol (CBD), tetrahydrocannabinol (THC), or cannabidiol (CBD) and tetrahydrocannabinol (THC). The Cannabis sativa-derived substances can be derived from all varieties of Cannabis sativa, including hemp. Cannabis varieties used for hemp production are one preferred source of CBD because of a high concentration of CBD with a low concentration of THC in materials from those plants.

Cannabis-derived compounds can be extracted from plant parts of Cannabis sativa, either from marijuana grade Cannabis or from industrial grade hemp. There are numerous Cannabis-derived compounds with the most prevalent being cannabinoids, such as cannabidiol (CBD) and tetrahydrocannabinol (THC), terpenes and flavonoids. CBD and THC are the best known of the cannabis-derived compounds and both have been studied for medical applications.

The cannabis-derived compounds are extracted from plant parts of Cannabis sativa. For use in the claimed invention, cannabis-derived compounds can be extracted from Cannabis plants having 0.3% tetrahydrocannabinol (THC) or less in the leaves and flowering heads (industrial hemp), from Cannabis plants having more than 0.3% tetrahydrocannabinol (THC) in the leaves and flowering heads, from the whole cannabis plant, or any combination thereof.

The cannabis-derived compounds can be extracted from the Cannabis plant parts using solvents such as butane, propane or ethanol, or by using a super-critical CO₂ extraction process. CBD can be derived from cannabis or hemp plant parts by extraction processes, including organic solvent extraction using one or more organic solvent, including, but not limited to, butane, propane and alcohols, with ethanol the preferred alcohol. Solvent-free CBD can be derived from cannabis or hemp plant parts by a super-critical CO₂ extraction process. CBD extracts useful for the claimed invention are in a liquid, oil or crystalline form.

Solvent directed processes, such as butane or propane extraction, must be purged of residual solvent prior to use. Ethanol is often used as an extraction solvent because the ethanol does not require purging to remove the ethanol, as do butane or propane. The composition of the extracts will vary based on the type of solvent used, with solvents such as butane and propane extracting only lipid soluble compounds, while alcohols extract both lipid and water-soluble compounds. Extracts produced by solvent extraction typically have an oily or waxy consistency and may require additional processing to remove impurities.

Supercritical CO₂ extraction uses phase changes in carbon dioxide, utilizing temperature and pressure, to produce an extract having a high purity that does not require purging to remove solvent residues. Because CO₂ does not leave residues in the extracted materials, the super-critical CO₂ extraction process yields an extract with greater than 99% purity that is free of solvent residues. Extracts produced by CO₂ extraction are a white needle-like crystalline powder.

THC can also be extracted from cannabis or hemp plant parts by extraction using an organic solvent, such as butane, propane or ethanol, or by the super-critical CO₂ extraction process. Additionally, solvents such as polyethylene glycol 400 (PEG 400), glycerin, or oelic acid can be used instead of or in addition to the butane, propane and ethanol solvent processes. Oils are the most commonly available form for THC extracts.

Terpenes and flavonoids are cannabis-derived compounds that can also be extracted from cannabis plant parts using butane, propane, ethanol or CO₂ extraction processes. Terpenes are aromatic compounds and flavonoids are phytonutrients, and these cannabis-derived compounds can be used as isolates or in combination with the CBD and/or THC cannabinoids. Terpenes and flavonoids work synergistically with cannabinoids to produce variations in benefits and side effects associated with the cannabinoids.

Cannabinoids other than CBD and THC can be derived from Cannabis sativa and may also be used in the disclosed invention. Most of the other cannabinoids are non-psychoactive or mildly psychoactive, and many have physiological effects similar to the effects seen for CBD. These other cannabinoids may work synergistically to enhance the effects of CBD and THC.

Additional cannabinoids that may be used in the disclosed invention include, but are not limited to: Cannabigerol CBG), Cannabidiolic Acid CBDA), Tetrahydrocannabinolic Acid (THCA), Cannabinol (CBN), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol monomethyl ether (CBGM), Cannabielsoin (CBE), Cannabicitran (CBT), and combinations thereof. Whole plant extracts of Cannabis sativa may also be used in the disclosed invention.

CBD and THC extracts can be used in combination in the nanosized phospholipid vesicle composition. CBD and THC are extracted from cannabis plants as separate isolates, either as oils or crystals, and then combinations of these extracts can be encapsulated in the phospholipid vesicles. Whole plant cannabis extracts can also be encapsulated in the nanosized phospholipid vesicles. Whole plant cannabis extract is an oily or waxy material that is soluble in lipids and alcohols. Whole plant extracts will vary in the amount of cannabinoids in the extract depending on whether the extract is prepared from marijuana-grade cannabis or industrial-grade hemp. Both types of whole plant extracts will have cannabinoids, terpenes and flavonoids, with the primary difference being the concentrations of CBD and THC in the final extract.

Nano-encapsulated CBD has a greater bioavailability than the equivalent, non-encapsulated form of a CBD substance, thus reducing the need for frequent re-medication, while providing a greater bioavailability, greater active duration and a greater ease of use resulting in increased compliance. That is, one way to enhance the bioavailability of CBD and other cannabinoids is to use nano-encapsulation of the CBD formulation using phospholipid vesicles.

Phospholipid vesicles are very similar to structures that the body naturally produces with phospholipid vesicles being tiny hollow spheres that have the ability to carry both water soluble and fat-soluble compounds through the body and cell membranes. Encapsulating cannabinoids in this manner would allow the cannabinoids to enter the bloodstream at a much greater bioavailability than a non-encapsulated form of the same therapeutic compounds. Phospholipid nano-encapsulation has shown promise in enhancing the speed of absorption as well as overall bioavailability of Cannabis sativa-derived substances.

A single dose of CBD in its nano-encapsulated form (using transdermal and sublingual administration methods) will reach the bloodstream faster, have a higher peak availability, and remain in the bloodstream longer, than its non-encapsulated counterpart. Nano-encapsulation technology is safe and has been for used successfully for topical drug delivery and oral nutritional delivery in human subjects. The nano-encapsulation technique enhances the bioavailability, uptake, and sustainability of the cannabinoids in the body.

Multiple routes of administration have been used for Cannabis sativa-derived substances with transmucosal and transdermal routes preferred for cannabinoids nanoencapsulated in phospholipid vesicles. A CBD amount of ˜10 mg is generally considered a “starting dose” by the medical community, with amounts typically going up to 300 mg daily for specific disease states. Cannabis sativa-derived substances which are nanoencapsulated in phospholipid vesicles can be administered in dosages ranging from 1 mg to 300 mg per day in single or divided doses. Optionally, dosages range from 10 mg to 100 mg per day in single or divided doses.

Disclosed herein is a composition comprising one or more Cannabis sativa-derived substance nanoencapsulated in phospholipid vesicles having phospholipids, ethanol and water. The phospholipid vesicles having the encapsulated Cannabis sativa-derived substance are sized from 25 nm to 200 nm. Phosphatidylcholine comprises at least 50% by weight of the phospholipids in the phospholipid vesicles. The concentration range of Cannabis sativa-derived substance encapsulated in the phospholipid vesicles is from 0.01% to 1.0% w/w.

Cannabis-derived extracts have enhanced bioavailability when encapsulated in nanosized phospholipid vesicles prior to administration to a subject as compared to non-encapsulated cannabinoids. Also disclosed herein is a method of using the composition having nano-encapsulated cannabis-derived compounds to administer the cannabis-derived active compounds to a subject in need of treatment. Cannabis-derived substances encapsulated in nanosized phospholipid vesicles can be advantageously administered through transmucosal and transdermal routes.

A delivery option comprising encapsulating one or more cannabis-derived compounds as passenger molecules in a potentiated nanolipidic process capable of delivering the cannabis-derived compound to a subject is disclosed. The route of delivery can be oral transmucosal (including sublingual and buccal) or transdermal.

Disclosed herein is a composition formulated for transmucosal delivery of Cannabis sativa derived substances encapsulated in a phospholipid delivery system. Cannabis sativa-derived substances for encapsulation are selected from cannabidiol (CBD), tetrahydrocannabinol (THC), Cannabis sativa-derived terpenes, Cannabis sativa-derived flavonoids, a whole plant extract of Cannabis sativa, and combinations thereof. Oral delivery can be via a transmucosal preparation, a sublingual/buccal preparation, or a preparation administered to the oral cavity. Transmucosal preparations can be liquids such as drops, sprays, aerosols or shots, gels, pastes, lozenges, gums, gummy candies, hard candies, orally dissolving strips, tablets, swish and swallow preparations, or other form suitable for contact to oral mucosa. The preparation may be applied to the mucosa under the tongue (sublingual), inside the cheeks (buccal), or placed inside the oral cavity.

Also disclosed herein is a composition formulated for transdermal delivery of Cannabis sativa derived substances encapsulated in a phospholipid delivery system. Cannabis sativa-derived substances for encapsulation are selected from cannabidiol (CBD), tetrahydrocannabinol (THC), Cannabis sativa-derived terpenes, Cannabis sativa-derived flavonoids, a whole plant extract of Cannabis sativa, and combinations thereof. For transdermal administration, the phospholipid vesicles with the nanoencapsulated Cannabis sativa-derived substance are applied to the epidermis of a subject for transdermal uptake of the Cannabis sativa-derived substance. Transdermal delivery can be via a topically applied preparation, such as a cream, lotion, ointment, wax, topically applied spray, gel, balm, transdermal patch or other transdermal application means.

Disclosed is a method of nano-encapsulating a Cannabis sativa-derived substance including the steps of preparing a cannabis stock by dissolving a Cannabis sativa-derived substance in an ethanolic stock having phospholipids, ethanol and water; diluting the cannabis stock with a quantity of ethanol; combining the diluted cannabis stock with a quantity of purified water having a pH of 6-8; and mixing the diluted cannabis stock/water combination vigorously to encapsulate the Cannabis sativa derived substance in one or more phospholipid vesicle.

Cannabis sativa-derived substances for the disclosed encapsulation method are selected from cannabidiol (CBD), tetrahydrocannabinol (THC), Cannabis sativa-derived terpenes, Cannabis sativa-derived flavonoids, a whole plant extract of Cannabis sativa, and combinations thereof. The Cannabis sativa-derived substance may be cannabidiol (CBD). The Cannabis sativa-derived substance may be tetrahydrocannabinol (THC). The Cannabis sativa-derived substance may be a combination of cannabidiol (CBD) and tetrahydrocannabinol (THC). The Cannabis sativa-derived substance may be a whole plant extract of Cannabis sativa.

The ethanolic stock in the disclosed nano-encapsulation method has 75-89% ethanol, 10-20% phospholipids and 1-5% water (w/w). In the present method, phosphatidylcholine (PC) is at least 50% of the phospholipid total in the ethanolic stock. In the present method, the phospholipid vesicles with nanoencapsulated Cannabis sativa-derived substance are sized from 25 nm to 200 nm. In the present method, the cannabis stock has a concentration range from 1 part Cannabis sativa-derived substance to 10 parts ethanolic stock up to 10 parts Cannabis sativa-derived substance to 1-part ethanolic stock (w/w). In the present method, the quantity of ethanol combined with the cannabis stock is at a ratio of 1:1 to 1:10 parts of cannabis stock to ethanol (by volume). In the present method, the quantity of water combined with the diluted cannabis stock is at a ratio from 1:5 to 1:25 parts of diluted cannabis stock to water (by volume).

Disclosed is a method of preparing lipid vesicles nanoencapsulating Cannabis sativa derived substances comprising the steps of preparing a plurality of non-spherical phospholipid vesicles having nanoencapsulated cannabidiol (CBD) derived from Cannabis sativa by dissolving CBD in an ethanolic stock having 75-89% ethanol, 10-20% phospholipids and 1-5% water (w/w), diluting the CBD-ethanol stock with a quantity of ethanol, and mixing the diluted CBD-ethanol stock combination to incorporate the CBD in said plurality of non-spherical phospholipid vesicles, said vesicles sized less than 10 nm; adding a quantity of purified water having a pH of 6-8 to the plurality of non-spherical phospholipid vesicles; and mixing said plurality of non-spherical phospholipid vesicles vigorously with said quantity of water to encapsulate the CBD in spherical phospholipid vesicles sized from 25 nm to 200 nm. A non-cannabis substance may optionally be dissolved in the quantity of water prior to combining with the diluted cannabis stock.

Disclosed is a method of preparing lipid vesicles nanoencapsulating Cannabis sativa derived substances comprising the steps of preparing a first plurality of non-spherical phospholipid vesicles having nanoencapsulated cannabidiol (CBD) derived from Cannabis sativa by dissolving CBD in an ethanolic stock having 75-89% ethanol, 10-20% phospholipids and 1-5% water (w/w), diluting the CBD-ethanol stock with a quantity of ethanol, and mixing the diluted CBD-ethanol stock combination to incorporate the CBD in said first plurality of non-spherical phospholipid vesicles, said vesicles sized less than 10 nm; preparing a second plurality of non-spherical phospholipid vesicles nanoencapsulating a non-CBD Cannabis sativa derived substance by dissolving the non-CBD Cannabis sativa derived substance in an ethanolic stock having 75-89% ethanol, 10-20% phospholipids and 1-5% water (w/w), diluting the non-CBD cannabis-ethanol stock with a quantity of ethanol, and mixing the diluted non-CBD cannabis-ethanol stock combination to incorporate the non-CBD Cannabis sativa in said second plurality of non-spherical phospholipid vesicles, said vesicles sized less than 10 nm; combining said first plurality of non-spherical phospholipid vesicles with said second plurality of non-spherical phospholipid vesicles; adding a quantity of purified water having a pH of 6-8 to the combined first and second plurality of non-spherical phospholipid vesicles; and mixing said combined first and second plurality of non-spherical phospholipid vesicles vigorously with said quantity of water to encapsulate the CBD and non-CBD Cannabis sativa derived substances in spherical phospholipid vesicles sized from 25 nm to 200 nm. A non-cannabis substance may be dissolved in the quantity of water prior to combining with the diluted cannabis stock.

A method for administration of a Cannabis sativa-derived substance comprising the steps of: providing a preparation having one or more Cannabis sativa-derived substance, nanoencapsulated in one or more phospholipid vesicles having phospholipids, ethanol, and water, and having a Cannabis sativa-derived substance concentration of 0.01% to 1.0% w/w; and administering a quantity of the preparation having 1 mg to 100 mg of Cannabis sativa-derived substance to a subject in need of treatment. The Cannabis sativa-derived substance is selected from cannabidiol (CBD), tetrahydrocannabinol (THC), Cannabis sativa-derived terpenes, Cannabis sativa-derived flavonoids, a whole plant extract of Cannabis sativa, and combinations thereof. The Cannabis sativa-derived substance may be cannabidiol (CBD), tetrahydrocannabinol (THC) or a combination of cannabidiol (CBD) and tetrahydrocannabinol (THC).

A method for administration of a Cannabis sativa-derived substance in an intraoral transmucosal and/or transdermal delivery protocol is disclosed comprising the steps of: providing one or more phospholipid vesicles in a first phospholipid solution having 75-89% ethanol (w/w), 10-20% phospholipids (w/w), and 1-5% water (w/w), and encapsulating one or more Cannabis sativa-derived substances in the one or more phospholipid vesicles in the first phospholipid solution; the encapsulated substances in the phospholipid vesicles being an initial cannabis preparation. The initial cannabis preparation is combined with a predetermined amount of ethanol to produce an ethanol-diluted cannabis preparation having phospholipid vesicles being predominately sized less than 10 nm, the size of vesicles in the ethanol-diluted cannabis preparation being controlled predominately by the amount of ethanol added to the initial cannabis preparation.

In the disclosed method, the ethanol-diluted cannabis preparation is combined with a predetermined quantity of water to produce increased-sized phospholipid vesicles in an encapsulated cannabis preparation. The increased-sized phospholipid vesicles in the encapsulated cannabis preparation are predominately sized between 25-200 nm having a concentration of 0.01% to 1.0% of the Cannabis sativa-derived substance encapsulated therein. The size of the increased-sized phospholipid vesicles in the encapsulated cannabis preparation is controlled predominately by the amount of water added to the ethanol-diluted cannabis preparation. The encapsulated cannabis preparation, in an unrefined form without vesicle size reduction by mechanical processing, is combined with one or more excipient ingredients suitable for intraoral transmucosal or transdermal application to yield a cannabis formulation for intraoral transmucosal or transdermal administration and administered as a quantity of the cannabis formulation having 1 mg to 100 mg of encapsulated Cannabis sativa-derived substances to a subject in need of treatment. In the disclosed method, the Cannabis sativa-derived substance may be selected from cannabidiol (CBD), tetrahydrocannabinol (THC), Cannabis sativa-derived terpenes, Cannabis sativa-derived flavonoids, a whole plant extract of Cannabis sativa, and combinations thereof. Optionally, the Cannabis sativa-derived substance is cannabidiol (CBD), tetrahydrocannabinol (THC), or a combination of cannabidiol (CBD) and tetrahydrocannabinol (THC). In the disclosed method, Cannabis sativa-derived substances are combined with the first phospholipid solution in a concentration range from 1 part Cannabis sativa-derived substance to 10 parts of the first phospholipid solution to 10 parts Cannabis sativa-derived substance to 1 part first phospholipid solution. The predetermined quantity of ethanol combined with the initial cannabis preparation has a ratio of 1:1 to 1:10 parts of initial cannabis preparation to ethanol. The predetermined quantity of water combined with the ethanol-diluted cannabis preparation has a ratio of 1:5 to 1:25 parts of ethanol-diluted cannabis preparation to water. phosphatidylcholine (PC) is at least 50% of the phospholipid total in the first phospholipid solution.

In the disclosed method, the preparation having one or more Cannabis sativa-derived substance nanoencapsulated in one or more phospholipid vesicles may be applied to an oral mucosa area of the subject and the preparation is kept in contact with the oral mucosa for 1 minute to 10 minutes or the preparation having one or more Cannabis sativa-derived substance nanoencapsulated in one or more phospholipid vesicles may be applied to an epidermal area of the subject and the preparation is kept in contact with the epidermal area for 1 minute to 24 hours. Transmucosal preparations can be liquids such as drops, sprays, aerosols or shots, gels, pastes, lozenges, gums, gummy candies, hard candies, orally dissolving strips, tablets, swish and swallow preparations, or other form suitable for contact to oral mucosa. The preparation may be applied to the mucosa under the tongue (sublingual), inside the cheeks (buccal), or placed inside the oral cavity. Transdermal delivery can be via a topically applied preparation, such as a cream, lotion, ointment, wax, topically applied spray, gel, balm, transdermal patch or other transdermal application means.

A method is disclosed for intraoral transmucosal administration of a Cannabis sativa-derived substance comprising the steps of: providing a preparation having one or more Cannabis sativa-derived substance selected from cannabidiol (CBD), tetrahydrocannabinol (THC), Cannabis sativa-derived terpenes, Cannabis sativa-derived flavonoids, a whole plant extract of Cannabis sativa, and combinations thereof, nanoencapsulated in one or more phospholipid vesicles, said one or more phospholipid vesicle having phospholipids, ethanol and water; applying a quantity of the preparation having from 1 mg to 100 mg to the subject's oral mucosa; and allowing the preparation to remain in contact with the mucosa for a period of time ranging from 1 minute to 10 minutes or may be kept in contact for 1 minute to 5 minutes. The preparation may be applied to intraoral mucosa, including sublingual and buccal mucosa. The preparation of nanosized phospholipid vesicles has a Cannabis sativa-derived substance concentration of 0.01% to 1.0% w/w, the phospholipid vesicles with nanoencapsulated Cannabis sativa-derived substance are sized from 25 nm to 200 nm, and phosphatidylcholine (PC) is at least 50% of the phospholipid total in the phospholipid vesicles.

Also disclosed herein is method for intraoral transmucosal administration of a Cannabis sativa-derived substance having the steps of: providing one or more phospholipid vesicles in a first phospholipid solution having 75-89% ethanol (w/w), 10-20% phospholipids (w/w), and 1-5% water (w/w), and encapsulating one or more Cannabis sativa-derived substances in the one or more phospholipid vesicles in the first phospholipid solution, the encapsulated substances in the phospholipid vesicles being an initial cannabis preparation, wherein the Cannabis sativa-derived substances are combined with the first phospholipid solution in a concentration range from 1 part Cannabis sativa-derived substance to 10 parts of the first phospholipid solution to 10 parts Cannabis sativa-derived substance to 1 part first phospholipid solution. The initial cannabis preparation is combined with a predetermined amount of ethanol to produce an ethanol-diluted cannabis preparation having phospholipid vesicles being predominately sized less than 10 nm, the predetermined quantity of ethanol combined with the initial cannabis preparation having a ratio of 1:1 to 1:10 parts of initial cannabis preparation to ethanol, the size of vesicles in the ethanol-diluted cannabis preparation being controlled predominately by the amount of ethanol added to the initial cannabis preparation.

In the disclosed method, the ethanol-diluted cannabis preparation with a predetermined quantity of water to produce increased-sized phospholipid vesicles in an encapsulated cannabis preparation, the size of the increased-sized phospholipid vesicles in the encapsulated cannabis preparation being controlled predominately by the amount of water added to the ethanol-diluted cannabis preparation. The encapsulated cannabis preparation in an unrefined form without vesicle size reduction by mechanical processing, is combined with one or more excipient ingredients suitable for intraoral transmucosal application to yield a cannabis formulation for intraoral transmucosal administration, and a quantity of the cannabis formulation having 1 mg to 100 mg of encapsulated Cannabis sativa-derived substances is administered to an intraoral mucosa of a subject in need of treatment. In the disclosed method, the increased sized phospholipid vesicles in the encapsulated cannabis preparation are predominately sized between 25-200 nm. The Cannabis sativa-derived substance is selected from cannabidiol (CBD), tetrahydrocannabinol (THC), Cannabis sativa-derived terpenes, Cannabis sativa-derived flavonoids, a whole plant extract of Cannabis sativa, and combinations thereof. The predetermined quantity of water combined with the ethanol-diluted cannabis preparation has a ratio of 1:5 to 1:25 parts of ethanol-diluted cannabis preparation to water, and the phospholipid vesicles in the encapsulated cannabis preparation have a concentration of 0.01% to 1.0% of the Cannabis sativa-derived substance encapsulated therein. The cannabis formulation is applied to the intraoral mucosa as a liquid drop, liquid spray, aerosol, liquid shot, gel, paste, lozenge, gum, gummy candy, hard candy, orally dissolving strip, tablet, or swish and swallow preparation.

A method for transdermal administration of a Cannabis sativa-derived substance comprising the steps of: providing a preparation of a Cannabis sativa-derived substance nano-encapsulated in one or more phospholipid vesicle having phospholipids, ethanol and water; applying a quantity of the preparation having from 1 mg to 100 mg to an area of the subject's epidermis; and allowing the preparation to remain in contact the epidermis for a period of time ranging from 1 minute to 24 hours. The Cannabis sativa-derived substance applied to the epidermis may be cannabidiol (CBD), tetrahydrocannabinol (THC), or a combination of cannabidiol (CBD) and tetrahydrocannabinol (THC). The preparation is applied via a cream, lotion, ointment, wax, gel, balm, spray or transdermal patch.

Also disclosed herein is a method for transdermal administration of a Cannabis sativa-derived substance comprising the steps of: providing one or more phospholipid vesicles in a first phospholipid solution having 75-89% ethanol (w/w), 10-20% phospholipids (w/w) with phosphatidylcholine (PC) being at least 50% of the phospholipid total in the first phospholipid solution, and 1-5% water (w/w), and encapsulating one or more Cannabis sativa-derived substances selected from cannabidiol (CBD), tetrahydrocannabinol (THC), Cannabis sativa-derived terpenes, Cannabis sativa-derived flavonoids, a whole plant extract of Cannabis sativa, and combinations thereof, in one or more phospholipid vesicles in the first phospholipid solution, the encapsulated substances in the phospholipid vesicles being an initial cannabis preparation. The initial cannabis preparation is combined with a predetermined amount of ethanol to produce an ethanol-diluted cannabis preparation having phospholipid vesicles being predominately sized less than 10 nm, the size of vesicles in the ethanol-diluted cannabis preparation being controlled predominately by the amount of ethanol added to the initial cannabis preparation. The ethanol-diluted cannabis preparation is combined with a predetermined quantity of water to produce increased-sized phospholipid vesicles in an encapsulated cannabis preparation, the increased sized phospholipid vesicles in the encapsulated cannabis preparation being predominately sized between 25-200 nm, and the size of the increased-sized phospholipid vesicles in the encapsulated cannabis preparation being controlled predominately by the amount of water added to the ethanol-diluted cannabis preparation. The encapsulated cannabis preparation in an unrefined form without vesicle size reduction by mechanical processing, is combined with one or more excipient ingredients suitable for transdermal application to yield a cannabis formulation for transdermal administration, and a quantity of the cannabis formulation having 1 mg to 100 mg of encapsulated Cannabis sativa-derived substances is administered to an epidermal area of a subject in need of treatment.

In the disclosed method, Cannabis sativa-derived substance is cannabidiol (CBD), tetrahydrocannabinol (THC), or a combination of cannabidiol (CBD) and tetrahydrocannabinol (THC). The Cannabis sativa-derived substance are combined with the first phospholipid solution in a concentration range from 1 part Cannabis sativa-derived substance to 10 parts of the first phospholipid solution to 10 parts Cannabis sativa-derived substance to 1 part first phospholipid solution, and the phospholipid vesicles in the encapsulated cannabis preparation have a concentration of 0.01% to 1.0% of the Cannabis sativa-derived substance encapsulated therein. The predetermined quantity of ethanol combined with the initial cannabis preparation has a ratio of 1:1 to 1:10 parts of initial cannabis preparation to ethanol. The predetermined quantity of water combined with the ethanol-diluted cannabis preparation has a ratio of 1:5 to 1:25 parts of ethanol-diluted cannabis preparation to water. The cannabis formulation is applied to the epidermis in a cream, lotion, ointment, wax, topically applied spray, gel, balm or transdermal patch.

A method is disclosed for intraoral transmucosal administration of a Cannabis sativa-derived substance comprising the steps of: providing a preparation having one or more Cannabis sativa-derived substance selected from cannabidiol (CBD), tetrahydrocannabinol (THC), Cannabis sativa-derived terpenes, Cannabis sativa-derived flavonoids, a whole plant extract of Cannabis sativa, and combinations thereof, nanoencapsulated in one or more phospholipid vesicles, said one or more phospholipid vesicle having phospholipids, ethanol and water; applying a quantity of the preparation having from 1 mg to 100 mg to the subject's oral mucosa; and allowing the preparation to remain in contact with the mucosa for a period of time ranging from 1 minute to 10 minutes or may be kept in contact for 1 minute to 5 minutes. The preparation may be applied to intraoral mucosa, including sublingual and buccal mucosa. The preparation of nanosized phospholipid vesicles has a Cannabis sativa-derived substance concentration of 0.01% to 1.0% w/w, the phospholipid vesicles with nanoencapsulated Cannabis sativa-derived substance are sized from 25 nm to 200 nm, and phosphatidylcholine (PC) is at least 50% of the phospholipid total in the phospholipid vesicles.

A method for transdermal administration of a Cannabis sativa-derived substance comprising the steps of: providing a preparation of a Cannabis sativa-derived substance nano-encapsulated in one or more phospholipid vesicle having phospholipids, ethanol and water; applying a quantity of the preparation having from 1 mg to 100 mg to an area of the subject's epidermis; and allowing the preparation to remain in contact the epidermis for a period of time ranging from 1 minute to 24 hours. The Cannabis sativa-derived substance applied to the epidermis may be cannabidiol (CBD), tetrahydrocannabinol (THC), or a combination of cannabidiol (CBD) and tetrahydrocannabinol (THC). The preparation is applied via a cream, lotion, ointment, wax, gel, balm, spray or transdermal patch.

To prepare the nanoencapsulated cannabinoids, the cannabis-derived extracts are first dissolved in an ethanolic stock comprising ethanol, phospholipids and water. Phospholipids are combined with ethanol and stirred at ambient room temperature until dissolved. Optionally, the ethanol used in the ethanolic stock is dehydrated 190 proof ethanol. A small quantity of water having a pH of 6-8 is added to the ethanol/phospholipid mixture and stirred until an optically clear ethanolic stock is formed. The ratio of the components in the ethanolic stock has a range of 75-89% ethanol, 10-20% phospholipids and 1-5% water, with a preferred ratio of 80% ethanol/17.75% phospholipids/2.25% water (w/w). Optionally, the phospholipids are derived from soy lecithin. The phospholipids are selected from phosphatidylcholine (PC), phosphatidylethanolamine (PE), phosphatidic acid (PA) and phosphatidylinositol (PI), and mixtures thereof. Optionally, phosphatidylcholine (PC) comprises at least 50% by weight of the phospholipid mixture. Optionally, phosphatidylcholine comprises 65% of the phospholipid total.

A cannabis stock is prepared by combining the ethanolic stock with one or more cannabis-derived extract and mixed at ambient room temperature until the extract is completely dispersed in the ethanolic stock. The ratio range of cannabis-derived extract to ethanolic stock is from 1 part cannabis-derived extract to 20 parts ethanolic stock to 20 parts cannabis-derived extract to 1-part ethanolic stock. Optionally, the ratio range of cannabis-derived extract to ethanolic stock is from 1 part cannabis-derived extract to 10 parts ethanolic stock to 10 parts cannabis-derived extract to 1 parts ethanolic stock. In one embodiment, the ratio of cannabis-derived extract to ethanolic stock is 1:1. Optionally, the ratio of cannabis-derived extract to ethanolic stock is any ratio between 0.5 g:4.5 g and 4.5 g:0.5 g. In one embodiment, the ratio of cannabis-derived extract to ethanolic stock is 1:1. In another embodiment, the ratio of cannabis-derived extract to ethanolic stock is 0.5 g:4.5 g. In another embodiment, the ratio of cannabis-derived extract to ethanolic stock is 4.5 g:0.5 g. Mixing of cannabis-derived extract into the ethanolic stock may be with a vortex, a magnetic stir-bar, an overhead mixer or a propeller type mixer.

Following preparation of the cannabis stock, a quantity of ethanol is added to the cannabis stock and thoroughly mixed at room temperature. Optionally, the additional quantity of ethanol is mixed with the cannabis stock using a vortex, a magnetic stir bar, or an overhead mixer. This dilution with ethanol can range from 1:1 to 1:100 by volume, with 1 to 100 parts of ethanol added to 1 part of the cannabis stock, inclusive of any amount between 1 and 100 parts of ethanol being added to 1 part of the cannabis stock. Optionally, the cannabis stock is combined with the ethanol at a ratio of 1:25 by volume. Optionally, the cannabis stock is combined with the ethanol at a ratio of 1:10 by volume. Optionally, the ethanol is added to the cannabis stock with the ethanol at 1:5 by volume. Optionally, the cannabis stock is combined with the ethanol at a ratio of 1:2.5 by volume. Optionally, the cannabis stock is combined with the ethanol at a ratio of 1:1 by volume. The secondary addition of ethanol adjusts the size of the lipid vesicles with the vesicle size decreasing as the amount of ethanol added increases. Following the addition of the secondary quantity of ethanol, the vesicles are less than 10 nm diameter and have not attained a spherical shape.

A quantity of an aqueous solvent comprising distilled or deionized water having a pH of 6-8 is added to the ethanol-diluted cannabis stock and thoroughly mixed at ambient room temperature until a uniform composition having an opalescent appearance is achieved. Optionally, the aqueous solvent is mixed with the ethanol-diluted cannabis stock using a vortex, a magnetic stir bar, or an overhead mixer.

This addition of aqueous solvent can range from 1:1 to 1:100 by volume, with 1 to 100 parts of aqueous solvent added to 1 part of the ethanol-diluted cannabis stock, inclusive of any amount between 1 and 100 parts of aqueous solvent being added to the ethanol-diluted cannabis stock. Optionally, the ratio is from 1:1 to 1:25 parts of ethanol-diluted cannabis stock to aqueous solvent. Optionally, the ratio is from 1:5 to 1:25 parts of ethanol-diluted cannabis stock to aqueous solvent. Optionally, the ratio is 1:9 parts of ethanol-diluted cannabis stock to aqueous solvent.

Secondary passenger substances can be dissolved in the aqueous solvent prior to combining with ethanol-diluted cannabis stock. Optionally, secondary passengers are water soluble cannabis-derived extracts. Optionally, the secondary passengers are non-cannabis substances. Non-cannabis substances are substances that can be used advantageously to enhance the usability of the nanosized phospholipid vesicles. Optionally, the substances enhance usability of the nanosized phospholipid vesicles as flavoring agents, masking agents, penetrating agents, augmenting agents, synergistic agents, or other agents to improve application and bioavailability of the encapsulated passenger.

Spherical nanosized phospholipid vesicles result in the opalescent composition when the aqueous solvent is fully combined with the ethanol-diluted cannabis stock. The opalescent composition has spherical nanosized phospholipid vesicles that are sized from 25 nm to 200 nm in diameter. Optionally, the vesicles are sized from 80 nm to 100 nm. Optionally, the vesicles are about 80 nm in diameter.

Cannabinoid compounds incorporated in the ethanol-diluted cannabis-derived extract/ethanolic stock have a concentration range of 0.1%40% (w/w). Following 1:10 dilution with the aqueous solvent, the nanosized phospholipid vesicles have a concentration of 0.01% to 1.0% (w/w). Optionally, the concentration of cannabinoid compounds incorporated in the ethanol-diluted cannabis-derived extract/ethanolic stock is from 1% to 2%. Optionally, following 1:10 dilution with the aqueous solvent, the nanosized phospholipid vesicles have a cannabinoid compound concentration from 0.1% to 0.2% (w/w).

A cannabis stock having CBD is prepared by combining a crystalline CBD isolate having greater than 99% purity with ethanolic stock at a concentration range of 0.1%-10% (w/w) and mixed at ambient room temperature until completely dissolved. The cannabis stock is then combined with a quantity of ethanol, 1:1 to 1:100 (by volume) and stirred until the mixture is homogenous. The ethanol-diluted cannabis stock is then combined with a quantity of aqueous solvent, 1:1 to 1:100 (by volume) and mixed until a uniform composition having an opalescent appearance is achieved. The opalescent composition has nanosized spherical phospholipid vesicles that are from 25 nm to 200 nm in diameter. Optionally, the vesicles are sized from 80 nm to 100 nm. Optionally, the vesicles are about 80 nm in diameter.

A cannabis stock having THC is prepared by combining THC oil at a concentration range of 0.1%-10% (w/w) with ethanolic stock and mixed at ambient room temperature until completely dissolved. The cannabis stock is then combined with a quantity of ethanol, 1:1 to 1:100 (by volume), and stirred until the mixture is homogenous. The ethanol-diluted cannabis stock is combined with a quantity of water, 1:1 to 1:100 (by volume), and mixed until a uniform composition having an opalescent appearance is achieved. The opalescent composition has nanosized spherical phospholipid vesicles that are from 25 nm to 200 nm in diameter. Optionally, the vesicles are sized from 80 nm to 100 nm. Optionally, the vesicles are about 80 nm in diameter.

Combinations of CBD and THC, at a concentration range of 0.1%-10% (w/w) of combined cannabinoids, can be co-encapsulated in the nanosized phospholipid vesicles such that a combination composition results. A cannabis stock is prepared by adding CBD and/or THC separately or together to the ethanolic stock and mixed at ambient room temperature until completely dissolved. The CBD/THC cannabis stock combination is then mixed with a quantity of ethanol, 1:1 to 1:100 (by volume), and further mixed until homogeneous. The ethanol-diluted cannabis stock is then combined with a quantity of aqueous solvent, 1:1 to 1:100 (by volume), and mixed until a uniform composition having an opalescent appearance is achieved. The opalescent composition has nanosized spherical phospholipid vesicles that are from 25 nm to 200 nm in diameter. Optionally, the vesicles are sized from 80 nm to 100 nm. Optionally, the vesicles are about 80 nm in diameter.

Optionally, the CBD and THC are combined and encapsulated together in a single addition to the ethanolic stock. Optionally, the CBD and THC are encapsulated separately in the ethanolic stock prior to the addition of the additional ethanol and the vesicles having CBD are combined with the vesicles having THC after addition of the additional ethanol to the individual preparations. Optionally, the CBD and THC encapsulation vesicles are combined prior to the addition of the aqueous solvent. Optionally, the CBD and THC vesicles are combined after addition of the aqueous solvent. The combining of the vesicles with two separately encapsulated materials advantageously allows for tuning of the ratios of the two compounds for specific applications as well as allowing for the maximum concentration of each compound to be individually encapsulated prior to combining.

In another embodiment, the THC is encapsulated as a lipophilic passenger added to the ethanolic stock and mixed with additional ethanol, and water-soluble CBD is added to the aqueous solvent phase, then THC/ethanolic stock/ethanol is combined with the CBD/aqueous solvent to form the final vesicles having both THC and CBD encapsulated therein.

A whole plant cannabis stock can be prepared by combining whole plant cannabis extracts at a concentration range of 0.1%-10% (w/w) with ethanolic stock and mixed at ambient room temperature until completely dissolved. The whole plant cannabis stock is then combined with a quantity of ethanol, 1:1 to 1:100 (by volume) and mixed until homogenous. The ethanol-diluted cannabis stock is combined with a quantity of aqueous solvent, 1:1 to 1:100, (by volume) and mixed until a uniform composition having an opalescent appearance is achieved. Optionally, the whole plant extract is a pourable oil, a viscous oil, or a waxy oil. Optionally, the whole plant extract is gently heated to liquefy the oils prior to combining with the ethanolic stock. The opalescent composition has nanosized spherical phospholipid vesicles that are from 25 nm to 200 nm in diameter. Optionally, the vesicles are sized from 80 nm to 100 nm. Optionally, the vesicles are about 80 nm in diameter.

Delivery options comprise encapsulating one or more cannabis-derived compounds as passenger molecules in a potentiated nanolipidic process capable of delivering the cannabis-derived compound to a subject. Optionally, the route of delivery is oral, via a transmucosal preparation, a sublingual preparation, a buccal preparation, or an oral preparation. Optionally, the delivery route is transdermal via a topically applied preparation, such as a topically applied cream, lotion, ointment, wax, topically applied spray, gel, balm, transdermal patch or other transdermal application means.

Cannabis sativa-derived substances which are nanoencapsulated in phospholipid vesicles can be administered in dosages ranging from 1 mg to 300 mg per day in single or divided doses or any dose between 1 mg and 300 mg per day. Optionally, dosages range from 10 mg to 100 mg per day in single or divided doses. Optionally, the dosages range from 1 mg to 20 mg per day in a single or divided dose.

Nanoencapsulated cannabidiol (CBD) for transmucosal delivery is disclosed. The nanoencapsulated lipid vesicles are formulated for transmucosal delivery of the cannabis-derived compounds to the mucosa of a subject. Optionally, the mucosa is oral, sublingual or buccal mucosa.

A method of encapsulating cannabis-derived compounds for transmucosal delivery is disclosed. The ethanolic stock for preparing the encapsulated nanolipidic preparation is manipulated by dilution with a non-aqueous solvent comprising ethanol and further manipulated by dilution with an aqueous solvent comprising water to achieve the desired transmucosal dose.

Disclosed herein is a formulation for transmucosal delivery of Cannabis sativa derived substances in a phospholipid delivery system. The substances can be derived from all varieties of Cannabis sativa, including hemp. Cannabis varieties used for hemp production are one preferred source of CBD because of a high concentration of CBD with a low concentration of THC in materials from those plants.

Cannabidiol can be extracted from hemp plant materials by a supercritical carbon dioxide extraction process, wherein phase changes induced in the CO₂, utilizing temperature and pressure, yield extracts free of toxic solvents. Other organic solvents, such as ethanol, butane, and propane, can also be used to extract Cannabis-derived substances, including cannabidiol (CBD), tetrahydrocannabinol (THC), flavonoids and terpenes. The transmucosal preparation is formulated for application to a subject as oral transmucosal, buccal or sublingually-applied liquids (as drops, sprays, aerosols or shots), gels, pastes, lozenges, gums, gummy candies, hard candies, orally dissolving strips, tablets, swish and swallow preparations or other transmucosal application means. Optionally, flavoring or masking agents are added to the preparation prior to administration. The preparation may be applied to the mucosa under the tongue (sublingual), inside the cheeks (buccal), or placed inside the oral cavity. Flavoring or masking agents can be combined with the aqueous solvent used in preparing the phospholipid vesicles.

Disclosed herein is a formulation for transdermal delivery of Cannabis sativa derived substances in a phospholipid delivery system. The substances can be derived from all varieties of Cannabis sativa, including hemp. Cannabis varieties used for hemp production are one preferred source of CBD because of a high concentration of CBD with a low concentration of THC in materials from those plants.

Cannabidiol can be extracted from hemp plant materials by a supercritical carbon dioxide extraction process, wherein phase changes induced in the CO₂, utilizing temperature and pressure, yield extracts free of toxic solvents. Other organic solvents, such as ethanol, butane and propane can also be used to extract Cannabis-derived substances, including cannabidiol (CBD), tetrahydrocannabinol (THC), flavonoids and terpenes. The transdermal preparation is formulated for application to skin as a cream, lotion, ointment, wax, topically applied spray, gel, balm, transdermal patch or other transdermal application means.

Single cannabinoid isolates, such as CBD and THC, can be encapsulated in nanosized phospholipid vesicles for transmucosal or transdermal delivery to a subject. Preparation of a composition of cannabidiol for transmucosal administration is described.

CBD from the Cannabaceae family, preferably Cannabis sativa, can be extracted from the Cannabis plant material by a supercritical CO₂ extraction process. Cannabidiol (CBD) is procured in a crystalline form having greater than 99% purity. Crystalline CBD useful for encapsulation is a fine white powder having needlelike crystals. While the supercritical CO₂ extraction process is preferred, ethanol and butane extractions are also commonly used.

Crystalline CBD having greater than 99% purity is combined with an ethanolic stock having phospholipids and water in an ethanol base until uniformly distributed in the ethanolic stock. Phospholipids used in the ethanolic stock are optionally derived from lecithin. Phospholipids used in the ethanolic stock are optionally derived from soy. When the cannabidiol is fully incorporated into the ethanolic stock, additional ethanol is added to the mixture. A quantity of water is then added to the mixture resulting in discrete nano-sized lipid vesicles having CBD encapsulated in a phospholipid vesicle. Optionally, flavoring or masking agents are combined with the quantity of water before mixing with the cannabidiol-phospholipid mixture.

The CBD concentration in the combined stock ranges from 0.1% to 10%. Optionally, the concentration range is from 1% to 5%. Optionally, the concentration range is from 1.167% to 2.0%. The encapsulated CBD concentration following 1:10 dilution with the aqueous solvent ranges from 0.01% to 1.0%. Optionally, encapsulated CBD concentration following 1:10 dilution with the aqueous solvent ranges from 0.1% to 0.5%. Optionally, the encapsulated CBD concentration following 1:10 dilution with the aqueous solvent ranges from 0.167% to 0.2%.

Table 1 shows a representation of percentages of components in a composition with encapsulated CBD.

TABLE 1
CBD Encapsulation Composition

| Code | Description | INCI | % w/w |
|---|---|---|---|
| A DZ0060 | Ethanolic Stock | Alcohol, Lecithin, Water | 0.833 |
| A CBD (hemp) | Cannabidiol | Cannabidiol | 0.167 |
| B DZ2330 | Ethanol | Alcohol | 9.00 |
| C DZ1370 | Purified Water | Water | 90.00 |
| | | | 100.00 |

Preparations of nanoencapsulated CBD were tested for stability. Results are summarized in Table 2 below.

TABLE 2
Summary of CBD Encapsulation Studies for 1CB
CBD (~99%) Crystalline Stability Studies

| Study | Vendor | | *Final CBD | | | | | | Fill size |
|---|---|---|---|---|---|---|---|---|---|
| Name/Start | of ~99% | Product | Concentration | Day 0 | Day 1 | Day 2 | Day 3 | | and |
| Date | CBD Crystals | Code | (mg/g) | (PSA) | (PSA) | (PSA) | (PSA) | Batch Size | Packaging |
| Study 2 | Isodiol | 1CB | ~1.65 mg/g | (N/A) | (~170 nm) | (N/A) | (~167 nm) | 5 grams | 5 g of 1CB |
| (25° C.) | (Lot#ISOB99109) | | | | | | | of CBD | in a 15 mL |
| (Jun. 27, 2017) | | | | | | | | produced | Centrifuge |
| | | | | | | | | and filled | Tube |
| | | | | | | | | into 1 | |
| | | | | | | | | centrifuge | |
| | | | | | | | | tube | |
| Study 2 | | 1CB | | (N/A) | (~185 nm) | (N/A) | (~178 nm) | 5 grams | |
| (4° C.) | | | | | | | | of CBD | |
| (Jun. 27, 2017) | | | | | | | | produced | |
| | | | | | | | | and filled | |
| | | | | | | | | into 1 | |
| | | | | | | | | centrifuge | |
| | | | | | | | | tube | |

| | | | | | | |
|---|---|---|---|---|---|---|
| Study 14B | Isodiol ISO99 | 1CB | ~1.65 mg/g | Day 0 Study: 1CB lasted up to 48 hrs | 150 grams | ~27 g |
| (Jan. 31, 2018) | (Lot#010918) | | | Day 7 Study: 1CB lasted up to 24 hrs | of CBD | of 1CB |
| | | | | Day 14 Study: 1CB lasted up to 48 hrs | produced | in a 1 oz |
| | | | | Day 21 Study: 3 bottles of 1CB lasted | and filled | Cobalt Blue |
| | | | | up to 48 hrs, the other 3 bottles are | into 6 | Glass Bottle |
| | | | | still in solution | Cobalt | |
| | | | | 33 Days later | Blue | |
| | | | | Day 35 Study: 1CB lasted up to 48 hrs | Bottles | |
| | | | | Day 56 Study: currently in progress | | |

*Final CBD Concentration dependent on purity of cannabidiol.
Combination Material Using a Whole Plant Extract haying ~70% Cannabinoids Stability Study

| | | | *Final | | | | | | |
|---|---|---|---|---|---|---|---|---|---|
| | | | Concentration | | | | | | |
| Study | | | of | | | | | | |
| Name/Start | | Product | Cannabinoids | Day 0 | Day 1 | Day 2 | Day 3 | Batch | Fill size and |
| Date | Material | Code | (mg/g) | (PSA) | (PSA) | (PSA) | (PSA) | Size | Packaging |
| Study 6 | **Whole plant | 1CB | ~1.17 mg/g | (113.1 nm) | (104.6 nm) | (67.9 nm) | (91.4 nm) | 5 grams | 5 g of 1CB |
| (Aug. 1, 2017) | Combo Material | | | | | | | produced | in a 15 mL |
| | | | | | | | | and filled | Centrifuge |
| | | | | | | | | into 1 | Tube |
| | | | | | | | | centrifuge | |
| | | | | | | | | tube | |
| Study 6B | **Whole plant | 1CB | ~1.17 mg/g | (124.5 nm) | (N/A) | (N/A) | (N/A) | 5 grams | 5 g of 1CB |
| (Aug. 4, 2017) | Combo Material | | | | | | | produced | in a 15 mL |
| | | | | | | | | and filled | Centrifuge |
| | | | | | | | | into 1 | Tube |
| | | | | | | | | centrifuge | |
| | | | | | | | | tube | |

Preparations were found to maintain stability from 24 hours up to more than one month.

### Example 1: Enhanced Serum Levels of Nano-Encapsulated Vs Non-Encapsulated CBD

For transmucosal administration, CBD was encapsulated in a nanosized phospholipid vesicle for the nanoencapsulated preparation, and for comparison purposes, CBD was combined with an olive oil carrier for a non-encapsulated control preparation. The nanoencapsulation vesicles were prepared with a shelf-stable ethanolic stock comprised of water, phospholipids and ethanol, or a similar solvent. Lecithin was used as a source of the phospholipids. The crystalline CBD for use as a control may be dispersed in olive oil, coconut oil or other orally acceptable oil.

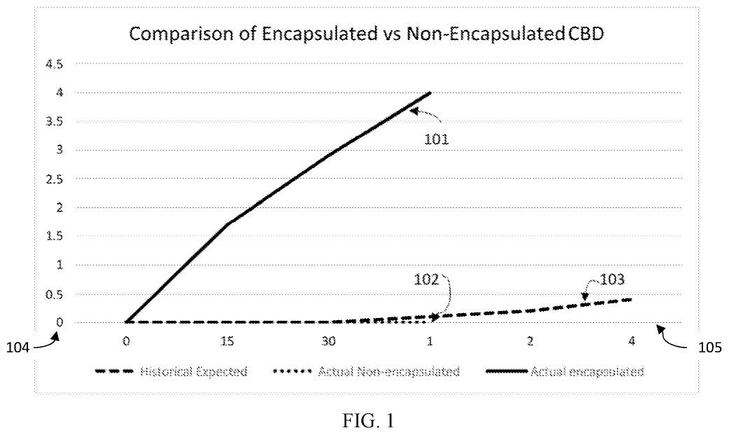

Cannabinoids have a greatly increased bioavailability when encapsulated in nanosized phospholipid vesicles as compared to non-encapsulated cannabinoids. FIG. 1 shows a comparison of serum levels achieved for oromucosal administration of cannabidiol encapsulated in nanosized phospholipid vesicles and a control of cannabidiol mixed in olive oil.

In the FIG. 1 graph, a comparison is shown of serum levels in mg/L (104) of encapsulated (101) vs. non-encapsulated (102) CBD against historical data (103) for CBD over time (105). Serum levels from subjects who received transmucosal administration of nano-encapsulated CBD (101) and serum levels from subjects who received transmucosal administration of non-encapsulated CBD in olive oil (102) were compared to historic expected results (103). These historic results for oromucosal bioavailability were compiled from multiple sources for plasma cannabinoid pharmacokinetics.

For the nano-encapsulated and non-encapsulated CBD comparison, test subjects in both groups were administered 1.65 mg CBD, sublingually, and blood samples were collected at regular time intervals and tested for levels of CBD. The FIG. 1 graph shows the serum level results obtained over the first four hours after administration for the nano-encapsulated CBD (101) and the non-encapsulated CBD (102).

For the historic expected results data (103), data was compiled for concentrations of 5, 10 and 15 mg/L and serum levels obtained showed less than one third of the CBD was found in the serum at the peak concentration (C max):
5 mg CBD→1.2 microgram per liter (nanogram per mL) C max;
10 mg CBD→3.0 microgram per liter (nanogram per mL) C max;
15 mg CBD→3.7 microgram per liter (nanogram per mL) C max.
(Tmax=3.6-4.5 hours and AUC (0-10.5 hours=4.1-12.6).

Surprisingly, the serum levels for the nano-encapsulated CBD (101) were not only higher than the historic results would have predicted, they demonstrated a greatly increased bioavailability of the encapsulated CBD over non-encapsulated CBD. The serum yield for a dosing of 1.65 mg CBD→3.99 microgram per liter (nanogram per mL) C 1 hour for the nanoencapsulated CBD. This demonstrates a ten-fold increase in serum concentration of nano-encapsulated CBD over the expected serum concentration of 0.4 microgram per liter as based on historical data for oromucosal bioavailability of CBD.

The serum levels for non-encapsulated CBD were much lower than expected. Based on the historical data, the expectation for non-encapsulated CBD administered in olive oil would be: 1.65 mg CBD→0.4 microgram per liter (nanogram per mL) C max. However, the actual results for non-encapsulated CBD (102) was: 1.65 mg CBD→0.0 microgram per liter (nanogram per mL) C 1 hour indicating that CBD in olive oil was not absorbed.

1. A method for administration of a Cannabis sativa-derived substance in an intraoral transmucosal and/or transdermal delivery protocol comprising the steps of:
providing one or more phospholipid vesicles in a first phospholipid solution having 75-89% ethanol (w/w), 10-20% phospholipids (w/w), and 1-5% water (w/w);
encapsulating one or more Cannabis sativa-derived substances in said one or more phospholipid vesicles in said first phospholipid solution, said encapsulated substances in said phospholipid vesicles being an initial cannabis preparation;
combining said initial cannabis preparation with a predetermined amount of ethanol to produce an ethanol-diluted cannabis preparation having phospholipid vesicles being predominately sized less than 10 nm, said size of vesicles in said ethanol-diluted cannabis preparation being controlled predominately by the amount of ethanol added to said initial cannabis preparation;
combining said ethanol-diluted cannabis preparation with a predetermined quantity of water to produce increased-sized phospholipid vesicles in an encapsulated cannabis preparation, said increased-sized phospholipid vesicles in the encapsulated cannabis preparation being predominately sized between 25-200 nm having a concentration of 0.01% to 1.0% of said Cannabis sativa-derived substance encapsulated therein, said size of said increased-sized phospholipid vesicles in said encapsulated cannabis preparation being controlled predominately by the amount of water added to said ethanol-diluted cannabis preparation;
combining said encapsulated cannabis preparation in an unrefined form without vesicle size reduction by mechanical processing, with one or more excipient ingredients suitable for intraoral transmucosal or transdermal application to yield a cannabis formulation for intraoral transmucosal or transdermal administration, and
administering a quantity of said cannabis formulation having 1 mg to 100 mg of encapsulated Cannabis sativa-derived substances to a subject in need of treatment.

2. The method of claim 1, wherein the Cannabis sativa-derived substance is selected from cannabidiol (CBD), tetrahydrocannabinol (THC), Cannabis sativa-derived terpenes, Cannabis sativa-derived flavonoids, a whole plant extract of Cannabis sativa, and combinations thereof.

3. The method of claim 2, wherein said Cannabis sativa-derived substance is cannabidiol (CBD), tetrahydrocannabinol (THC), or a combination of cannabidiol (CBD) and tetrahydrocannabinol (THC).

4. The method of claim 1 wherein said Cannabis sativa-derived substances are combined with said first phospholipid solution in a concentration range from 1 part Cannabis sativa-derived substance to 10 parts of the first phospholipid solution to 10 parts Cannabis sativa-derived substance to 1 part first phospholipid solution.

5. The method of claim 1, wherein said predetermined quantity of ethanol combined with said initial cannabis preparation has a ratio of 1:1 to 1:10 parts of initial cannabis preparation to ethanol.

6. The method of claim 1, wherein said predetermined quantity of water combined with said ethanol-diluted cannabis preparation has a ratio of 1:5 to 1:25 parts of ethanol-diluted cannabis preparation to water.

7. The method of claim 1, wherein phosphatidylcholine (PC) is at least 50% of the phospholipid total in the first phospholipid solution.

8. A method for intraoral transmucosal administration of a Cannabis sativa-derived substance comprising the steps of:
providing one or more phospholipid vesicles in a first phospholipid solution having 75-89% ethanol (w/w), 10-20% phospholipids (w/w), and 1-5% water (w/w);
encapsulating one or more Cannabis sativa-derived substances in said one or more phospholipid vesicles in said first phospholipid solution, said encapsulated substances in said phospholipid vesicles being an initial cannabis preparation, wherein said Cannabis sativa-derived substances are combined with said first phospholipid solution in a concentration range from 1 part Cannabis sativa-derived substance to 10 parts of the first phospholipid solution to 10 parts Cannabis sativa-derived substance to 1 part first phospholipid solution;
combining said initial cannabis preparation with a predetermined amount of ethanol to produce an ethanol-diluted cannabis preparation having phospholipid vesicles being predominately sized less than 10 nm, said predetermined quantity of ethanol combined with said initial cannabis preparation has a ratio of 1:1 to 1:10 parts of initial cannabis preparation to ethanol, said size of vesicles in said ethanol-diluted cannabis preparation being controlled predominately by the amount of ethanol added to said initial cannabis preparation;
combining said ethanol-diluted cannabis preparation with a predetermined quantity of water to produce increased-sized phospholipid vesicles in an encapsulated cannabis preparation, said size of said increased-sized phospholipid vesicles in said encapsulated cannabis preparation being controlled predominately by the amount of water added to said ethanol-diluted cannabis preparation;
combining said encapsulated cannabis preparation in an unrefined form without vesicle size reduction by mechanical processing, with one or more excipient ingredients suitable for intraoral transmucosal application to yield a cannabis formulation for intraoral transmucosal administration, and
administering a quantity of said cannabis formulation having 1 mg to 100 mg of encapsulated Cannabis sativa-derived substances to an intraoral mucosa of a subject in need of treatment.

9. The method of claim 8 wherein said increased sized phospholipid vesicles in the encapsulated cannabis preparation are predominately sized between 25-200 nm.

10. The method of claim 8 wherein the Cannabis sativa-derived substance is selected from cannabidiol (CBD), tetrahydrocannabinol (THC), Cannabis sativa-derived terpenes, Cannabis sativa-derived flavonoids, a whole plant extract of Cannabis sativa, and combinations thereof.

11. The method of claim 8 wherein said predetermined quantity of water combined with said ethanol-diluted cannabis preparation has a ratio of 1:5 to 1:25 parts of ethanol-diluted cannabis preparation to water.

12. The method of claim 8 wherein said phospholipid vesicles in said encapsulated cannabis preparation have a concentration of 0.01% to 1.0% of said Cannabis sativa-derived substance encapsulated therein.

13. The method of claim 8, wherein the cannabis formulation is applied to the intraoral mucosa as a liquid drop, liquid spray, aerosol, liquid shot, gel, paste, lozenge, gum, gummy candy, hard candy, orally dissolving strip, tablet, or swish and swallow preparation.

14. A method for transdermal administration of a Cannabis sativa-derived substance comprising the steps of:
providing one or more phospholipid vesicles in a first phospholipid solution having 75-89% ethanol (w/w), 10-20% phospholipids (w/w) with phosphatidylcholine (PC) being at least 50% of the phospholipid total in the first phospholipid solution, and 1-5% water (w/w);
encapsulating one or more Cannabis sativa-derived substances selected from cannabidiol (CBD), tetrahydrocannabinol (THC), Cannabis sativa-derived terpenes, Cannabis sativa-derived flavonoids, a whole plant extract of Cannabis sativa, and combinations thereof, in said one or more phospholipid vesicles in said first phospholipid solution, said encapsulated substances in said phospholipid vesicles being an initial cannabis preparation;
combining said initial cannabis preparation with a predetermined amount of ethanol to produce an ethanol-diluted cannabis preparation having phospholipid vesicles being predominately sized less than 10 nm, said size of vesicles in said ethanol-diluted cannabis preparation being controlled predominately by the amount of ethanol added to said initial cannabis preparation;
combining said ethanol-diluted cannabis preparation with a predetermined quantity of water to produce increased-sized phospholipid vesicles in an encapsulated cannabis preparation, said increased sized phospholipid vesicles in the encapsulated cannabis preparation being predominately sized between 25-200 nm, said size of said increased-sized phospholipid vesicles in said encapsulated cannabis preparation being controlled predominately by the amount of water added to said ethanol-diluted cannabis preparation;
combining said encapsulated cannabis preparation in an unrefined form without vesicle size reduction by mechanical processing, with one or more excipient ingredients suitable for transdermal application to yield a cannabis formulation for transdermal administration, and
administering a quantity of said cannabis formulation having 1 mg to 100 mg of encapsulated Cannabis sativa-derived substances to an epidermal area of a subject in need of treatment.

15. The method of claim 14 wherein said Cannabis sativa-derived substance is cannabidiol (CBD), tetrahydrocannabinol (THC), or a combination of cannabidiol (CBD) and tetrahydrocannabinol (THC).

16. The method of claim 14 wherein said Cannabis sativa-derived substance are combined with said first phospholipid solution in a concentration range from 1 part Cannabis sativa-derived substance to 10 parts of the first phospholipid solution to 10 parts Cannabis sativa-derived substance to 1 part first phospholipid solution.

17. The method of claim 14 wherein said phospholipid vesicles in said encapsulated cannabis preparation have a concentration of 0.01% to 1.0% of said Cannabis sativa-derived substance encapsulated therein.

18. The method of claim 14 wherein said predetermined quantity of ethanol combined with said initial cannabis preparation has a ratio of 1:1 to 1:10 parts of initial cannabis preparation to ethanol.

19. The method of claim 14 wherein said predetermined quantity of water combined with said ethanol-diluted cannabis preparation has a ratio of 1:5 to 1:25 parts of ethanol-diluted cannabis preparation to water.

20. The method of claim 14 wherein the cannabis formulation is applied to the epidermis in a cream, lotion, ointment, wax, topically applied spray, gel, balm or transdermal patch.
